(19) United States
(12) Patent Application Publication
(10) Pub. No.: US 2026/0166130 A1
(43) Pub. Date: Jun. 18, 2026

(54) PEPTIDE COMPOSITION HAVING ANGIOGENIC ACTIVITY, AND PHARMACEUTICAL COMPOSITION USING SAME AND COSMETIC COMPOSITION USING SAME

(71) Applicants: LABTOMEDI CRO CO.,LTD., Seoul (KR); THE KOREA INSTITUTION FOR PUBLIC SPERM BANK, Busan (KR)

(72) Inventors: Bo Sun JOO, Busan (KR); Ji Myung CHOI, Busan (KR)

(73) Assignees: LABTOMEDI CRO CO.,LTD., Seoul (KR); THE KOREA INSTITUTION FOR PUBLIC SPERM BANK, Busan (KR)

(21) Appl. No.: 18/852,074

(22) PCT Filed: Sep. 21, 2022
(86) PCT No.: PCT/KR2022/014071

§ 371 Date: Sep. 27, 2024

(30) Foreign Application Priority Data
Mar. 28, 2022 (KR) 10-2022-0037997

(51) Int. Cl. A61K 38/45; A61K 8/66; A61K 38/19; A61P 9/00; A61P 17/02; A61Q 19/00

(52) U.S. Cl. CPC A61K 38/45; A61K 8/66; A61K 38/19; A61P 9/00; A61P 17/02; A61Q 19/00; C12Y 204/02012

(57) ABSTRACT

The present invention relates to a peptide composition having angiogenic activity, and a pharmaceutical composition using same and a cosmetic composition using same. The peptide forms a hydrogen bond with nicotinamide mononucleotide, which is a natural ligand of visfatin, and has high angiogenic activity, and thus can be used in pharmaceutical and cosmetic compositions for increasing angiogenic activity. The peptide composition having angiogenic activity, according to the present invention, contains, as active ingredients, a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y), or a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V). The first peptide and the second peptide form hydrogen a bond with nicotinamide mononucleotide and the first peptide and the second peptide form a bond with GLY384, ARG196 and ARG311, which are active site amino acids of a nicotinamide mononucleotide.

### TECHNICAL FIELD

The present invention relates to a peptide composition having angiogenic activity, a pharmaceutical composition comprising the same, and a cosmetic composition comprising the same, wherein the peptide composition contains a peptide based on the active site of visfatin, wherein the peptide has high angiogenic activity and thus may be used as a pharmaceutical composition and a cosmetic composition for increasing angiogenic activity.

### BACKGROUND ART

The formation of the vascular system can be classified into vasculogenesis, which refers to the process by which blood vessels are formed in our body, and angiogenesis, which refers to the de novo formation of blood vessels from pre-existing blood vessels. Angiogenesis is known to be an essential process for supplying blood for tumor growth and metastasis, but it is also an essential process in various normal physiological phenomena such as embryogenesis, tissue repair, and organ regeneration (Hoeben et al., 2004). In addition, angiogenesis plays a very important role in skin regeneration, including wound healing and tissue recovery [Tonnesen et al., 2000; Li et al., 2003)], and in physiological events such as follicle development and embryo implantation (Geva and Jaffe, 2000; Devesa and Caicedo, 2019; Billhaq et al., 2020).

The skin is likely to age or to be damaged by exposure to physical stimulation and trauma such as burns. Currently, various procedures such as facelift, micro-fat grafting, laser and platelet rich plasma therapies, and stem cell culture and transplantation are being used to treat skin aging and damage, but a fundamental therapeutic method has not yet been established. In addition, although a therapeutic strategy using adipose tissue-derived stem cells has been applied (Gaur et al., 2017; Cho et al., 2018; Huayllani et al., 2020), there are many difficulties in using the same in general clinical practice, due to difficulties such as invasive collection of adipose tissue and space and time for culture.

Recently, based on the phenomenon of skin regeneration observed in embryonic stages of some amphibians and rodents, and even humans, a skin regeneration-based therapeutic strategy for skin aging and skin damage that considers the interaction of growth factors involved in the wound healing process has emerged (Moore et al., 2018; Stoica et al., 2020), and the importance of angiogenesis in skin regeneration has been highlighted (Lu et al., 2021). As such, angiogenesis plays a very important role in skin regeneration.

Since most natural peptide sequences act as agonists on their receptors, natural peptides are considered as a starting point to find designed small molecule peptide sequences with functional activity. Protein-peptide docking simulation is a computer-aided drug design (CADD) technique and is very useful in deriving small-molecule peptides that can act identically or similarly to natural ligands and produce biological efficacies at least similar to that of natural peptides when bound to receptors (Lee et al., 2015).

Vascular endothelial growth factor (VEGF) and visfatin are known as representative regulatory factors of angiogenesis. Visfatin has a molecular weight of 55 kDa and was originally known as a pre-B cell colony enhancing factor that promotes the growth of B lymphocytes precursors (Samal et al., 1994), but has recently been identified as a type of adipokine secreted from various cells such adipocytes, macrophages, and amniotic epithelial cells (Ognjanovic et al., 2001; Fukuhara et al., 2005; Curat et al., 2006). Many researchers, including our research team, have reported that visfatin not only stimulates the expression of VEGF, but also stimulates angiogenesis by itself (Adya et al., 20081; Xiao et al., 2009; Bae et al., 2009, Choi et al., 2012).

However, visfatin has a high molecular weight, which significantly limits its development as a therapeutic agent for promoting skin regeneration or new blood vessel formation. In contrast, low-molecular-weight peptides have advantages such as high physiological activity, high specificity, stability, and diversity of structures and molecules, and thus the utility and marketability of the peptides in the pharmaceutical market have recently expanded (Fosgerau & Hoffmann, 2015). Therefore, if low-molecular-weight proteins or peptides with angiogenesis-promoting effects is developed, their development potential and clinical utility as preventive and therapeutic agents for skin aging and damage are expected to further increase.

Therefore, the present invention is intended to develop peptides with angiogenesis-promoting effects, based on the active site of visfatin by using protein-peptide docking simulation that is a computer-aided drug design technique.

### DISCLOSURE

### Technical Problem

The present invention has been made in order to solve the above-described problems, and an object of the present invention is to develop a peptide having angiogenic activity, based on the active site of visfatin using protein-docking computer simulation, since visfatin with a molecular weight of 55 kDa has limitations in developing as a therapeutic agent due to its high molecular weight.

The technical problems to be solved by the present invention are not limited to the technical problems mentioned above, and other technical problems not mentioned can be clearly understood by those skilled in the art to which the present invention pertains from the following description.

### Technical Solution

In the present invention, protein-peptide docking simulation that is a computer-aided drug design technique has been used to solve the above-described problems and develop peptide-based substances with angiogenic activity.

A peptide composition having angiogenic activity according to the present invention may contain, as an active ingredient:
a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

The first peptide and the second peptide may form hydrogen bonds with nicotinamide mononucleotide, and
the first peptide and the second peptide may form bonds with GLY384, ARG196, and ARG311, which are active site amino acids of nicotinamide mononucleotide.

In addition, a pharmaceutical composition for activating angiogenesis according to the present invention may contain, as an active ingredient:
a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

In addition, a cosmetic composition for activating angiogenesis according to the present invention may contain, as an active ingredient:
a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

### Advantageous Effects

The present invention has been made in order to solve the problem of limitations in developing visfatin as a therapeutic agent, due to its high molecular weight. According to the above “Technical Solution”, it is possible to develop peptides with angiogenic activity, based on the active site of visfatin by protein-peptide docking simulation that is a computer-aided drug design technique.

These peptides may have angiogenesis-promoting effects that are equivalent to or better than that of visfatin.

### BRIEF DESCRIPTION OF DRAWINGS

FIG. 1 shows the nucleotide sequence of a peptide composition having angiogenic activity derived from visfatin according to the present invention. The sequence belonging to the B-domain of visfatin is shown in blue and red, and six nucleotide sequences represented by hotspots are shown in red.

FIG. 2 shows a pose in which nicotinamide mononucleotide (NMN) is docked to the active site of visfatin according to one example of the present invention. The NMN is drawn as a stick, and the visfatin active site residues are represented by balls and sticks. The blue-colored broken lines represent H-bond interactions.

FIG. 3 shows a pose in which nicotinamide mononucleotide (NMN) is docked to the active site of a first peptide according to one example of the present invention. The NMN is drawn as a stick, and the visfatin active site residues are represented by balls and sticks. The blue-colored broken lines represent H-bond interactions.

FIG. 4 shows a pose in which nicotinamide mononucleotide (NMN) is docked to the active site of a second peptide according to one embodiment of the present invention. The NMN is drawn as a stick, and the visfatin active site residues are represented by balls and sticks. The blue-colored broken lines represent H-bond interactions.

FIG. 5 shows the cytotoxic effects of the first peptide and the second peptide on HUVEC cells, evaluated according to one example of the present invention. Cell viability was estimated 24 hours after treatment with the first peptide and the second peptide using an MTT assay. Data are presented as mean values, and Vis in the figure represents visfatin.

FIG. 6 shows the cytotoxic effects of the first peptide and the second peptide on cell invasion, evaluated according to one example of the present invention. (A) shows representative images, and (B) is a graph of the invaded cells after 24 hours. HUVECS (2×10⁴) were seeded in 100 μL of serum-free medium with visfatin peptides added to the upper compartment of the transwell and the full medium was added to the lower compartment. After 24 hours, cell invasion was determined by counting the total number of cells in a single filter using optical microscopy (40×). Data are presented as mean±SD of three independent experiments. ***P<0.0001 (vs. control).

FIG. 7 shows the effects of the first peptide and the second peptide on cell migration, evaluated according to one example of the present invention. (A) shows representative images, and (B) is a graph of the migrated cells at 18 hours. HUVECs (2×10⁵/well) were seeded on 24-well plates and incubated overnight. Then, the cells were scratched using a P200 pipette tip and further incubated in media with or without the first peptide and the second peptide. The cells were allowed to migrate for 18 hours. Migration patterns were observed using a phase-contrast microscope (×40). Data are presented as mean±SD of three independent experiments. ***P<0.0001 (vs. control).

FIG. 8 shows the effects of the first peptide and the second peptide on tube formation in Matrigel assays according to one example of the present invention. (A) shows representative tube formation images, (B) shows a schematic image of angiogenesis, (C) shows the number of branches, and (D) shows the number of total branches. HUVECs (20,000 cells/well) were seed on a layer of previously polymerized Matrigel and treated with or without the first peptide and the second peptide. The Matrigel culture was incubated at 37° C. After 4 hours, changes in cell morphology were captured using a phase-contrast microscope (×40). Each sample was assayed in duplicate, and independent experiments were repeated three times. *P<0.05, **P,0.01, ***P<0.001 (vs. control).

### BEST MODE

The terms used in the present specification will be briefly explained, and the present invention will be described in detail.

The terms used in the present invention are currently widely used general terms selected in consideration of their functions in the present invention, but they may change depending on the intents of those skilled in the art, precedents, or the advents of new technology. Accordingly, the terms used in the present invention should be defined based on the meaning of the term and the entire contents of the present invention, rather than the simple term name.

Throughout the present specification, it is to be understood that when any part is referred to as “comprising” any component, it does not exclude other components, but may further comprise other components, unless otherwise specified.

Embodiments of the present invention will be described below in detail with reference to the accompanying drawings so that those skilled in the art can easily carry out the present invention. However, the present invention may be embodied in various different forms and is not limited to the embodiments described herein.

The specific details including the problems to be solved, the means for solving the problems, and the effects of the present invention are included in the embodiments described below and the drawings. The advantages and features of the present invention, and the way of attaining them, will become apparent with reference to the embodiments described below in conjunction with the accompanying drawings.

Hereinafter, the present invention will be described in more detail with reference to the accompanying drawings.

Angiogenesis plays a very important role in skin regeneration. Visfatin is a 55 kDa cytokine secreted from adipocytes, and not only stimulates the expression of vascular endothelial growth factor (VEGF), a regulator of angiogenesis, but also has an angiogenesis-promoting function. However, visfatin itself has a high molecular weight, which significantly limits its development as a therapeutic agent. Therefore, the present invention is intended to develop peptides with angiogenic activity, based on the active site of visfatin through a protein-peptide docking simulation method that is a computer-aided drug design method.

In the present invention, the B-domain (residues 181 to 390) with active site domain of visfatin was truncated into small peptides using the overlapping technique to generate visfatin-based peptides having efficacy similar or superior to visfatin. The truncated peptides were then subjected to molecular docking analysis using two protein-peptide docking programs (HADDOCK and GalaxyPepDock) to generate small peptides with the highest affinity for visfatin. Among them, two peptides with the highest affinity were investigated for their cytotoxicity using human umbilical vein endothelial cells, and then examined for their angiogenic activities, including cell migration, invasion, and blood vessel formation.

As a result, when the B-domain was truncated using the overlapping technique based on an amino acid length of 6 with 3 overlapping amino acids (dataset-1), an amino acid length of 7 with 2 overlapping amino acids (dataset-2), and an amino acid length of 9 with 3 overlapping amino acids (dataset-3), a total of 114 truncated peptides were obtained, including 38 peptides in dataset-1, 42 peptides in dataset-2, and 34 peptides in dataset-3. From the 114 peptides, 9 peptides (10 to 15 amino acid residues) with high affinity were derived through docking programs, and among them, two peptides with the highest affinity showed no cytotoxicity in the MTT assay and showed superior angiogenic activities, including cell migration, cell invasion, and blood vessel formation, compared to visfatin itself or the positive control fibroblast growth factor (FGF).

These results demonstrate that the peptides obtained through protein-peptide docking simulation have more efficient angiogenic activity than visfatin. Furthermore, these results suggest that protein-peptide docking simulation is very effective in generating low-molecular-weight peptides with the same or higher efficacy from high-molecular-weight proteins.

A peptide composition having angiogenic activity according to the present invention contains, as an active ingredient, a first peptide or a second peptide.

The first peptide consists of 10 amino acid residues, that is, leucine (L)-glutamic acid (E)-tyrosine (Y)-lysine (K)-leucine (L)-histidine (H)-aspartic acid (D)-phenylalanine (F)-glycine (G)-tyrosine (Y).

The second peptide consists of 12 amino acid residues, that is, glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

The first peptide and the second peptide may form hydrogen bonds with nicotinamide mononucleotide, and the first peptide and the second peptide may form bonds with GLY384, ARG196, and ARG311, which are active site amino acids of nicotinamide mononucleotide.

In addition, a pharmaceutical composition for activating angiogenesis according to the present invention may comprise, as an active ingredient, the peptide composition having angiogenic activity.

The pharmaceutical composition for activating angiogenesis comprises, as an active ingredient, a first peptide or a second peptide.

The first peptide consists of 10 amino acid residues, that is, leucine (L)-glutamic acid (E)-tyrosine (Y)-lysine (K)-leucine (L)-histidine (H)-aspartic acid (D)-phenylalanine (F)-glycine (G)-tyrosine (Y).

The second peptide consists of 12 amino acid residues, that is, glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

The first peptide and the second peptide may form hydrogen bonds with nicotinamide mononucleotide, and the first peptide and the second peptide may form bonds with GLY384, ARG196, and ARG311, which are active site amino acids of nicotinamide mononucleotide.

The pharmaceutical composition of the present invention may be formulated or used in combination with drugs such as antihistamines, anti-inflammatory analgesics, anticancer drugs, and antibiotics that are already in use. In pharmaceutical dosage forms, the composition of the present invention may be used in the form of pharmaceutically acceptable salts thereof, and may also be used alone or in appropriate association, as well as in combination, with pharmaceutically acceptable salts thereof.

The pharmaceutical composition according to the present invention may be formulated and used in the form of oral formulations such as powders, granules, tablets, capsules, suspensions, emulsions, syrups, and aerosols, preparations for external use, suppositories, and sterile injectable solutions, according to the respective conventional methods. Carriers, excipients and diluents that may be contained in the composition include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

The composition may be formulated with commonly used diluents or excipients, such as fillers, extenders, binders, wetting agents, disintegrants, surfactants, etc. Solid formulations for oral administration include tablets, pills, powders, granules, capsules and the like, and such solid formulations are prepared by mixing the peptide with at least one excipient, for example, starch, calcium carbonate, sucrose, lactose or gelatin. In addition to simple excipients, lubricants such as magnesium stearate or talc are also used. Liquid formulations for oral administration include suspensions, solutions, emulsions, and syrups, and may contain various excipients, for example, wetting agents, flavoring agents, aromatics and preservatives, in addition to water and liquid paraffin, which are frequently used simple diluents. Formulations for parenteral administration include sterilized aqueous solutions, non-aqueous solutions, suspensions, emulsions, freeze-dried preparations, and suppositories. As non-aqueous solvents or suspending agents, propylene glycol, polyethylene glycol, plant oils such as olive oil, injectable esters such as ethyl oleate, and the like may be used. As the base of the suppositories, witepsol, Macrogol, Tween 61, cacao butter, laurin fat, glycerogelatin and the like may be used.

The composition of the present invention may be administered to mammals, including rats, mice, livestock, and humans, through various routes. All modes of administration can be contemplated, and for example, the composition may be administered orally, intrarectally, or by intrarectal, intravenous, intramuscular, subcutaneous, intrauterine, intrathecal or intra-cerebrovascular injection.

The composition according to the present invention can be mixed with an injectable composition and administered in an injectable form to a mammalian site where angiogenesis is to be induced. The injectable composition is preferably in the form of an isotonic aqueous solution or suspension. The pharmaceutical composition may be sterilized or contain auxiliary agents such as preservatives, agents, emulsifiers, stabilizers, wetting solubilizers, salts for regulating osmotic pressure, and/or buffers, or other therapeutically useful substances.

Although the preferred dose of the pharmaceutical composition of the present invention varies depending on the patient's condition and body weight, the severity of disease, the form of drug, the route of administration, and the period of administration, it can be appropriately selected by a person skilled in the art. However, for a desirable effect, the composition of the present invention may be administered at a dose of 0.5 mg/kg to 10 mg/kg per day, and may be administered several times a day to once a week depending on the half-life. The above dose does not limit the scope of the present invention in any way.

In addition, a cosmetic for activating composition angiogenesis according to the present invention may comprise, as an active ingredient, the peptide composition having angiogenic activity.

The cosmetic composition for activating angiogenesis comprises, as an active ingredient, a first peptide or a second peptide.

The first peptide consists of 10 amino acid residues, that is, leucine (L)-glutamic acid (E)-tyrosine (Y)-lysine (K)-leucine (L)-histidine (H)-aspartic acid (D)-phenylalanine (F)-glycine (G)-tyrosine (Y).

The second peptide consists of 12 amino acid residues, that is, glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

The first peptide and the second peptide may form hydrogen bonds with nicotinamide mononucleotide, and the first peptide and the second peptide may form bonds with GLY384, ARG196, and ARG311, which are active site amino acids of nicotinamide mononucleotide.

The compositions according to the present invention may contain other ingredients, which may impart a synergistic effect to the main effect of the present invention, within a range that does not impair the main effect. For example, the compositions may further contain additives such as fragrances, colorants, bactericides, antioxidants, preservatives, humectants, thickeners, inorganic salts, emulsifiers, and synthetic polymer substances, as well as auxiliary components such as water-soluble vitamins, oil-soluble vitamins, high-molecular-weight peptides, high-molecular-weight polysaccharides, and seaweed extracts, in order to improve physical properties thereof. These components may be appropriately selected and added without difficulty by those skilled in the art depending on the formulation or intended use, and the amount thereof added may be selected within a range that does not impair the purpose and effect of the present invention, but may be 0.01 to 5 wt %, preferably 0.01 to 3 wt %, based on the total weight of the composition.

In addition, the formulation of the composition according to the present invention may also be in various forms, such as a solution, an emulsion, a viscous mixture, a tablet, a powder, etc., which may be administered in various ways, such as simple drinking, injection, spraying, or squeezing.

Hereinafter, the present invention will be described in more detail by way of examples. The purpose, features, and advantages of the present invention will be easily understood through the following examples. The present invention is not limited to the examples described herein and may be embodied in other forms. The examples disclosed herein are provided to fully convey the spirit of the present invention to those skilled in the art. Therefore, the present invention should not be construed as being limited by the following examples.

### Example 1: Materials and Methods

### 1-1) Construction of Peptide Library

The following systematic approach was used to determine significant hotspots and active spots in the amino acid sequence of visfatin. Three-dimensional structural analysis of visfatin showed that the active site was located in the B-domain (residues 181 to 390) (Kim et al., 2006; Zhang et al., 2011). The globular B-domain was truncated into small peptides using the overlapping technique based on amino acid lengths of 6, 7 and 9 with 3, 2 and 3 overlapping amino acids, respectively. The three-dimensional structures of the truncated peptide sequences were confirmed using the PEPstrMOD web server (Kaur et al., 2007; Singh et al., 2015), and then “structure-based molecular docking” simulation analysis considering the three-dimensional structure of the visfatin protein was performed to identify hotspots or active spots in the B-domain and design natural peptide sequences derived from visfatin.

### 1-2) Molecular Docking Simulation Analysis

The three-dimensional structure of visfatin (PDB ID 2G95) was obtained from the RCSB protein data bank (http:/www.rcsb.org), and the known amino acids in the active site of visfatin were confirmed to be TYR18, PHE193, TYR195, ARG196, GLY197, ASP219, HIS247, ARG311, ARG313, GLY353, and ASP354 (Kim et al., 2006). Then, the peptides with the highest affinity for visfatin were identified using two protein-peptide docking simulation programs, HADDOCK (Dominguez et al., 2003) and GalaxyPepDock (Lee et al., 2015).

### 1-3) Peptide Synthesis

The two synthetic peptides with the highest affinity, derived from the docking simulation, were obtained from GL Biochem Ltd. (Shanghai, China) and used for angiogenic activity assessment. The synthetic peptides were supplied as dry powders with a purity of 95% or more guaranteed by mass spectrometry and HPLC, reconstituted in dimethyl sulfoxide (DMSO) to a concentration of 1 mg/mL, and stored at −20° C. until use.

### 1-4) Culture of Human Vascular Endothelial Cells (HUVECs)

HUVECS (PromoCell, Heidelberg, Germany) were cultured in endothelial basal medium 2 (EBM) supplemented with Supplement Mix (PromoCell) containing 2% fetal bovine serum (FBS). The Supplement Mix consisted of 5 ng/mL epidermal growth factor (EGF), 10 ng/ml basic fibroblast growth factor (bFGF), 20 ng/mL insulin-like growth factor (IGF), 0.5 ng/mL VEGF, 1 ug/mL ascorbic acid, 22.5 ug/mL heparin, and 0.2 ug/mL hydrocortisone. In some experiments, cells were cultured in EBM medium supplemented with Supplement Mix, and cells between passages 2 and 7 were used in all experiments. Cells were cultured for 24 hours in a 5% CO₂ incubator at a 37° C.

### 1-5) MTT Cytotoxicity Assay

HUVECs were seeded at a density of 10,000 cells in 96-well plates (SPL, Korea) and incubated overnight with EBM-2 medium containing visfatin (1,000 ng/ml) and each of the peptides with the highest affinity, obtained from docking simulation, in a 5% CO₂ incubator at 37° C. MIT reagent (Sigma-Aldrich) was added to each well to a final concentration of 0.5 mg/ml. After 4 hours, the medium was removed, the formazan crystals formed in the cells were dissolved in dimethyl sulfoxide (DMSO), and the absorbance of the formazan solution was measured at a wavelength of 540 nm using a Synergy HTX Multi-Mode Reader (BIO-TEK, Vermont, USA). Each sample was assayed in triplicate.

### 1-6) Assessment of Cell Invasion Using Transwell

The invasion capacity of cells was determined using a 24-well transwell system. The upper side of the transwell membrane was coated with 1 mg/ml Matrigel at 10 UL/well. HUVECs (2×10⁴ cells) and serum-free EBM-2 medium (100 μL) supplemented with visfatin-derived peptides were placed on the upper compartment of the transwell and then filled with medium up to the bottom. The cells were incubated in a 5% CO₂ incubator at 37° C. for 24 hours, fixed with methanol, and stained with hematoxylin and eosin. The cells on the upper surface of the membrane were removed by wiping with a cotton swab. Cell invasion was determined by counting the total number of cells in a single filter using optical microscopy at 40× magnification. Each sample was assayed in duplicate, and independent experiments were repeated three times.

### 1-7) Cell Migration Assay

HUVECs were seeded in 24-well plates (SPL, Korea) at a density of 2×10⁵ cells and incubated with EBM medium overnight in a 5% CO₂ incubator at 37° C. Then, the cells were scratched using a P200 pipette tip to induce a wound. Subsequently, the cells were further incubated in EBM-2 medium supplemented with 1% FBS and the visfatin-derived peptides so as to allow the cells to migrate. The migration patterns were observed using a phase-contrast microscope and images were captured. The wound diameters were photographed at 16 to 24 hours. Wound closure was observed and photographed using optical microscopy at 40× magnification. Cell migration was quantified by counting the number of cells that had moved beyond the reference line.

### 1-8) Blood Vessel Formation Assay

HUVECs (20,000 cells/well) were seeded on a layer of previously polymerized Matrigel, treated with the visfatin-derived peptides, and then incubated in a 5% CO₂ incubator at 37° C. for 4 hours. Then, changes in the cell morphology were observed and photographed using a phase-contrast microscope at 40× magnification. Each sample was assayed in duplicate, and independent experiments were repeated three times. Angiogenesis was analyzed with ImageJ software using an angiogenesis analyzer (Bethesda, MD, USA).

### 1-9) Statistical Analysis

All data are presented as mean±standard deviation (SD). The experimental results were statistically processed using the Prism 9 program (GraphPad) by the t-test and two-way ANOVA. Statistical significance was set at P value<0.05.

### Example 2: Construction of Peptide Library Using Overlapping Technique

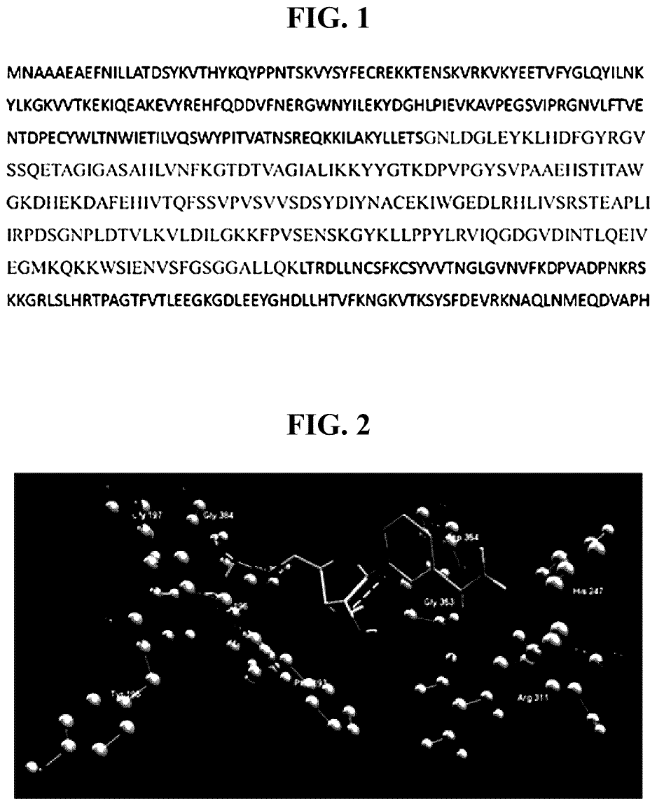

In the first step, to find a hotspot or active site in the visfatin sequence and obtain visfatin-based peptide sequences having a biological effect similar or superior to visfatin, as shown in FIG. 1, the B-domain (residues 181 to 390) of visfatin was truncated using the overlapping technique based on an amino acid length of 6 with 3 overlapping amino acids (dataset-1), an amino acid length of 7 with 2 overlapping amino acids (dataset-2), and an amino acid length of 9 with 3 overlapping amino acids (dataset-3). As a result, a total of 114 truncated peptides were obtained, including 38 peptides in dataset-1, 42 peptides in dataset-2, and 34 peptides in dataset-3.

### Example 3: Derivation of Small Molecule Peptides by Docking Simulation

Two protein-peptide docking simulation programs, HADDOCK and GalaxyPepDock, were used to calculate the binding affinity of the 114 truncated peptides for visfatin. The HADDOCK score shows the fit score between the peptide amino acid sequence and the visfatin active site, identified by the RCSB protein data bank, and the GalaxyPepDock score shows the interaction score between the peptide and visfatin based on the interaction scores between the ligand and receptor built in the database. Through these two docking simulation programs, 27 peptides with high affinity scores were obtained, and among them, a peptide with an amino acid sequence length of 9 and 3 overlapping amino acids (dataset-3) showed a higher affinity score. Therefore, a peptide sequence length of 9 was considered as the standard length, and six hotspots “LEYKLHDFGYRGVSSQ”, “GIALIKKYYGTKDPV”, “IYACEKIWGEDLRH”, “HSTITAWGK DHEKDAF”, “KFPVSENSKGYKLLPPY”, “GMKOKKWSIENVSFG” in the B-domain of visfatin, indicated in red in FIG. 1, were reconstructed to generate 76 peptides with different amino acid lengths.

To derive the peptide with the highest affinity for visfatin among the 76 peptides, docking simulations using HADDOCK and GalaxyPepDock were performed once more, and 9 peptides as shown in Table 1 below were finally derived. Docking simulations were performed on the interactions of nicotinamide mononucleotide (NMN), which is a natural ligand of visfatin, with the active site of visfatin, in order to examine whether the interactions of these nine peptides with visfatin would be similar to the interactions of visfatin with NMN.

TABLE 1

| | | | GalaxyPepDock |
|---|---|---|---|
| | Peptides | HADDOCK Score | Score |
| 1 | LEYKLHDFGY (10 AA) | −106.0 +/− 2.0 | 16.0 |
| 2 | EYKLHDFGYRGV (12 AA) | −105.9 +/− 3.5 | 17.0 |
| 3 | ALIKKYYGTKDPV (13 AA) | −111.2 +/− 2.9 | 10.0 |
| 4 | STITAWGKDHEKDAF | −124.3 +/− 6.5 | 1.0 |
| | (15AA) | | |
| 5 | ACEKIWGEDLRH (12 AA) | −85.5 +/− 5.5 | 0.0 |
| 6 | CEKIWGEDLRH (11 AA) | −93.6 +/− 4.2 | 0.0 |
| 7 | EKIWGEDLRH (10 AA) | −71.6 +/− 2.0 | 0.0 |
| 8 | GMKQKKWSIENVSF | −97.8 +/− 4.3 | 15.0 |
| | (14 AA) | | |
| 9 | ENSKGYKLLPPY (12 AA) | −82.7 +/− 2.9 | 18.0 |

As a result, as shown in FIG. 2, the active site amino acids, GLY384 (with an H-bond length (H-BL) of 2.52 Å), ARG196 (H-BLs 2.63 Å and 2.56 Å), and ARG311 (H-BL 2.63 Å) formed hydrogen bonds with NMN, signifying their importance in the catalytic activity. The interactions of the nine peptides with visfatin were similar to those of NMN with visfatin. These results suggest that the nine peptides may act as potential agonists of visfatin.

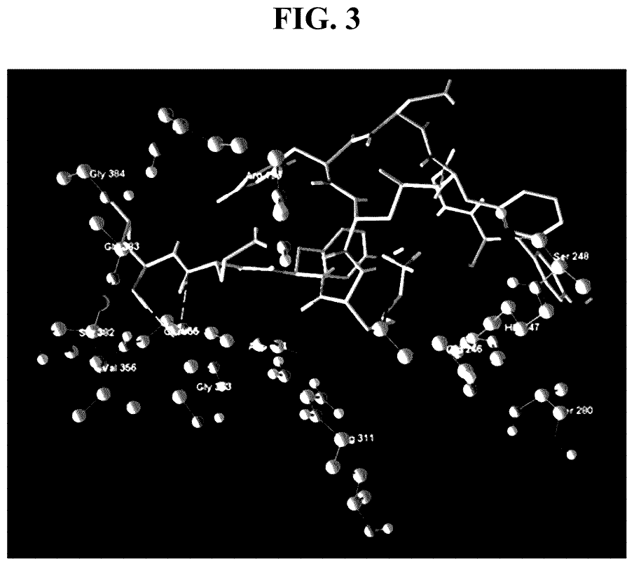

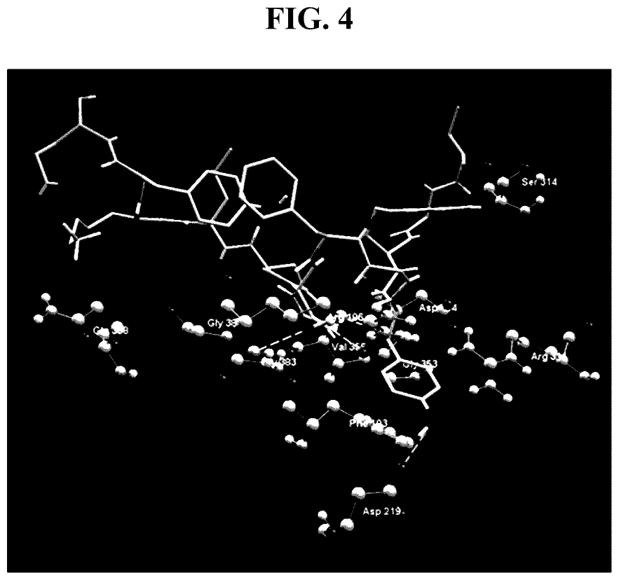

FIGS. 3 and 4 show the molecular interactions of the first peptide (LEYKLHDFGY, 10 amino acids) and the second peptide (EYKLHDFGY RGV, 12 amino acids) with the highest affinity among the nine peptides within the active site of visfatin.

### Example 4: Cytotoxic Effect of Visfatin-Derived Peptides

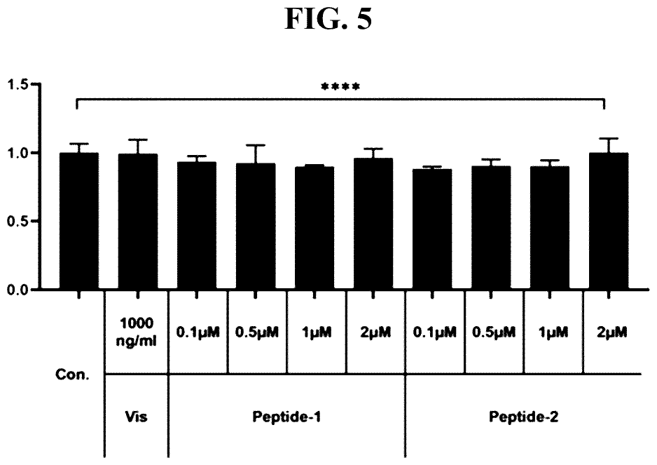

To investigate the effect of the two peptides (the first peptide and the second peptide) with the highest affinity for visfatin on the cell viability of HUVECs, each peptide was measured in an MIT assay at concentrations of 0.1 μM, 0.5 μM, 1.0 μM, and 2.0 μM. When the cells were treated with each peptide and incubated overnight, the two peptides did not affect the viability of the cells regardless of the treatment concentration, as shown in FIG. 5. These results suggest that the visfatin-derived peptides are not cytotoxic at concentrations of 0.1 μM to 2.0 μM.

### Example 5: Effect of Visfatin-Derived Peptides on Angiogenesis

To investigate the angiogenic activity of the first and second peptides with the highest affinity for the active site of visfatin, cell invasion, cell migration, and angiogenic tube formation on Matrigel were investigated.

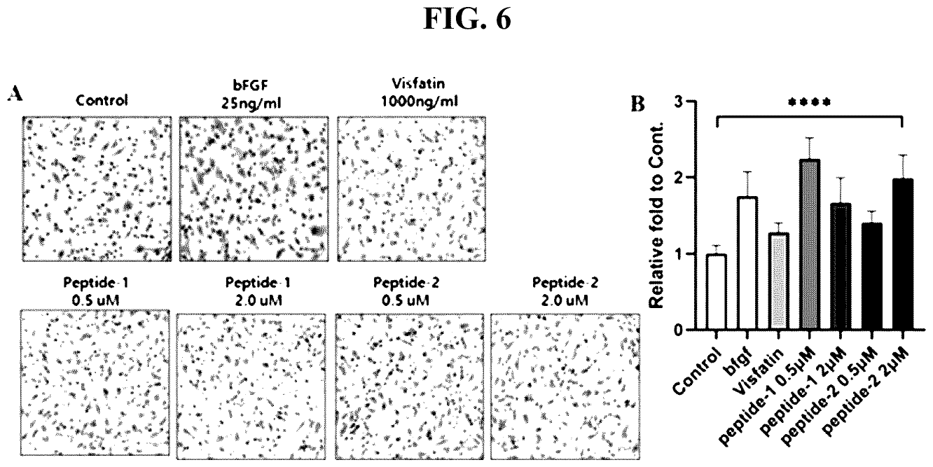

As shown in FIG. 6, when cells were treated with visfatin-derived peptides, both peptides increased cell invasion compared to the control group. In particular, the first peptide at a concentration of 0.5 UM or the second peptide at a concentration of 2.0 μM increased cell invasion by more than about 2-fold compared to the control group or the visfatin-treated group. This increase in cell invasion by the peptides was higher than that by bGFG used as the positive control.

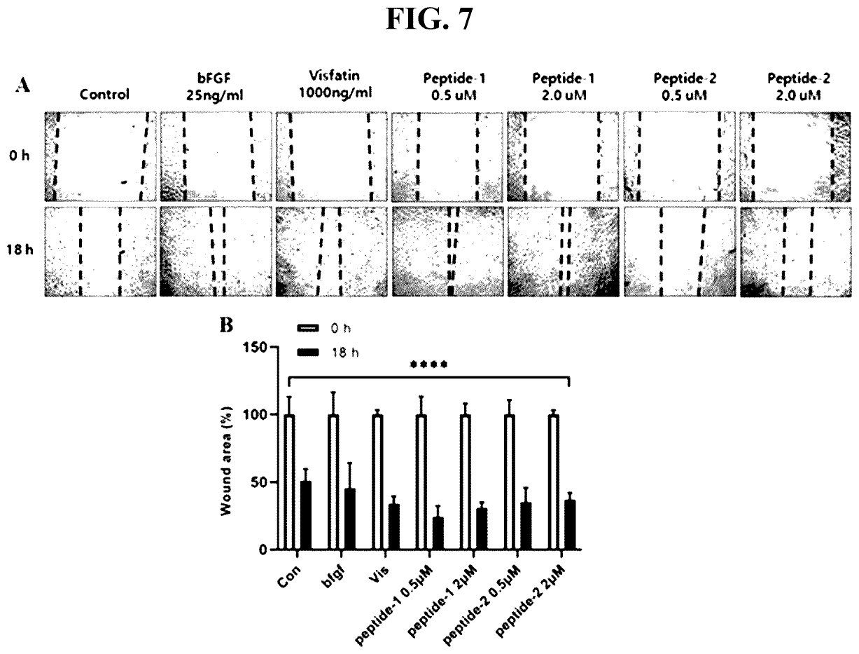

As shown in FIG. 7, treatment with the two peptides also resulted in a two-fold increase in cell migration at concentrations of 0.5 μM for the first peptide and 2.0 μM for the second peptide compared to the control group.

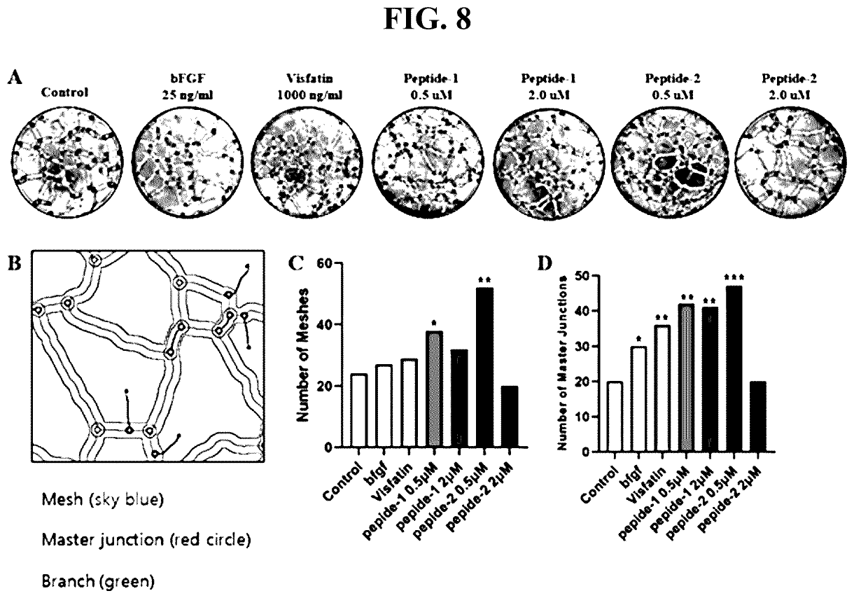

Angiogenic tube formation on Matrigel is an indication of the formation of blood vessels, such as capillaries, and is evaluation by two factors, mesh formation and master junction formation. The number of meshes was not significantly changed by treatment with b-FGF and visfatin used as positive controls, but was significantly increased by treatment with the first peptide and the second peptide at a concentration of 0.5 μM (P<0.01 and P<0.001, respectively). In contrast, as shown in FIG. 8, the number of master junctions was significantly increased not only bFGF and visfatin, but also in both the first peptide and the second peptide except at the 2.0 μM concentration of the second peptide.

The present invention has been made in order to solve the problem of limitations in developing visfatin, a 55 kDa cytokine secreted from adipocytes, as a therapeutic agent, due to its high molecular weight. According to the above “Technical Solution”, it is possible to develop peptides with angiogenic activity, based on the active site of visfatin.

It is possible to develop a peptide having angiogenesis-promoting effects equivalent to or better than to that of visfatin, based on the active site of visfatin.

The above description of the present invention is exemplary, and those of ordinary skill in the art to which the present invention pertains will appreciate that the present invention may be embodied in other specific forms without departing from the technical spirit or essential characteristics of the present invention.

Therefore, it should be understood that the embodiments described above are exemplary in all aspects and are not restrictive. Furthermore, the scope of the present invention is defined by the appended claims rather than the detailed description, and it should be understood that all modifications or variations derived from the meanings and scope of the present invention and equivalents thereto are included in the scope of the appended claims.

1. A peptide composition having angiogenic activity, containing, as an active ingredient:
a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

2. The peptide composition of claim 1, wherein the first peptide and the second peptide form hydrogen bonds with nicotinamide mononucleotide.

3. The peptide composition of claim 1, wherein the first peptide and the second peptide form bonds with GLY384, ARG196, and ARG311, which are active site amino acids of nicotinamide mononucleotide.

4. A pharmaceutical composition for activating angiogenesis, containing, as an active ingredient:
a first peptide consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).

5. A cosmetic composition for activating angiogenesis, containing, as an active ingredient:
a first peptide p consisting of leucine (L)-glutamic acid (E)-tyrosine (Y)-lysine (K)-leucine (L)-histidine (H)-aspartic acid (D)-phenylalanine (F)-glycine (G)-tyrosine (Y); or
a second peptide consisting of glutamic acid (E)-tyrosine (Y)-lysine (K)-leucine (L)-histidine (H)-aspartic acid (D)-phenylalanine (F)-glycine (G)-tyrosine (Y)-arginine (R)-glycine (G)-valine (V).
